(19) United States
(12) Patent Application Publication
(10) Pub. No.: US 2025/0051753 A1
(43) Pub. Date: Feb. 13, 2025

(54) PURIFICATION OF MACROMOLECULES

(71) Applicant: PHYNEXUS, INC., San Jose, CA (US)

(72) Inventors: Douglas GJERDE, San Jose, CA (US); Don APLAON, San Jose, CA (US)

(73) Assignee: PHYNEXUS, INC., San Jose, CA (US)

(21) Appl. No.: 18/723,715

(22) PCT Filed: Dec. 29, 2022
(86) PCT No.: PCT/US2022/054220

§ 371 Date: Jun. 24, 2024

Related U.S. Application Data
(60) Provisional application No. 63/296,224, filed on Jan. 4, 2022.

(30) Foreign Application Priority Data
Mar. 2, 2022 (EP) 22159731.3

(51) Int. Cl. C12N 15/10; C12N 15/85

(52) U.S. Cl. CPC C12N 15/101; C12N 15/85

(57) ABSTRACT

The present disclosure provides an automated method for chemical treatment of cells in a mixing chamber, wherein the automatable method comprises providing cells, providing a chemical agent for treating cells, forming a liquid mixture comprising the cells and the chemical agent, and mixing the liquid mixture by providing a back-and-forth liquid flow in the mixing chamber. An automated process for purifying macromolecules, such as plasmids, and an automated system comprising a workstation configured for purifying such macromolecules are also provided.

### TECHNICAL FIELD

The present invention relates to the field of purification of macromolecules as well as to automatable tools for use therein. More specifically, the present invention relates to an automated method for chemical treatment of cells in a mixing chamber as well as to an automated process for purifying macromolecules, such as plasmids. The invention also relates to a mixing process therein as such, as well as to devices and systems for use in purification of macromolecules and the like.

### BACKGROUND

One macromolecule which currently is of increasing commercial and scientific interest due to its usefulness as a vector of nucleic acid is a plasmid. The plasmid is a small, extrachromosomal DNA molecule within a cell that is physically separated from chromosomal DNA and can replicate independently of it. Although most plasmids are double-stranded DNA molecules, some consist of single-stranded DNA or predominantly double-stranded RNA. While chromosomes are large and contain all essential genetic information for living under normal conditions, plasmids are usually very small and contain only additional genes that may be useful in certain situations or conditions. Plasmids are commonly used bacterial cloning vectors for production of large amounts of desired proteins, and in the field of gene therapy a plasmid may be arranged e.g. to express a protein that is lacking in mammalian cells. Today, use of plasmids is highly relevant in vaccine production, for example where a genetically engineered plasmid containing a DNA sequence that encodes an antigen(s) against which an immune response is sought is introduced into a patient.

Another nucleic acid vector of great interest is a viral vector, that is a non-infectious submicroscopic agent. Viral vectors are widely used in gene therapy and cancer therapy as well as for development of vaccines. The three viruses most commonly used in the above-mentioned medical applications are adenovirus, adeno-associated virus and lentivirus. Viruses are diverse and display a large variety of genomic structures. The lentivirus is an enveloped retrovirus with a single-stranded RNA genome. The adeno-associated virus is nonenveloped virus with a single-stranded DNA genome. The adenovirus is a nonenveloped virus with a double-stranded DNA genome. The particularly useful feature of the above viruses is their potential to infect both dividing and nondividing cells. Further advantages of the use of the lentivirus and the adenovirus include ease of handling and their ability to bear larger DNA inserts. Furthermore, the adenovirus has been used for delivery of CRISPR/Cas9 gene editing systems and for producing viral vector COVID-19 vaccines.

Another vehicle for transportation of macromolecules from one cell to another is exosomes and other extracellular vesicles. Exosomes are membrane-bound extracellular vesicles that are produced in the endosomal compartment of most eukaryotic cells. Since exosomes have the ability to elicit potent cellular responses in vitro and in vivo, exosomes are increasingly being recognized as potential therapeutics. For example, exosomes offer distinct advantages that uniquely position them as highly effective drug carriers. Further, exosomes can be considered a promising carrier for effective delivery of small interfering RNA, and patient-derived exosomes have been employed as a novel cancer immunotherapy in several clinical trials.

In plasmid purification, as ingredients are mixed, the genomic DNA may be sheared and at least in part recovered together with purified plasmid DNA, thereby rendering the product useless at least for pharmaceutical preparations. Therefore, there is a difficulty in finding such a treatment of the cells containing plasmids of interest that it would be both effective for plasmid release and not cause shearing of genomic DNA.

In manually controlled procedures, ingredients may be combined to form a mixture, and then a carrier encompassing the mixture, such as a bottle or a tube, may be subjected to gentle inversion in view of achieving a uniform mixture. A dye may be added to the mixture for monitoring the efficacy of the mixing process, thus the mixing process may be stopped when the desired efficacy is achieved. The above may limit the duration of the process to a minimum and thereby reduce the degree of plasmid shearing.

Currently proposed automated methods for plasmid purification include mixing streams, agitating a stream or paddles. All of these methods have limitations and drawbacks including causing possible genomic shearing. Paddle mixing provides for high shear velocities at the end of the paddle Stream mixing, which relies on turbulent mixing in a tube or a chamber, may also involve shearing. Thus, prior art devices are difficult to integrate into a fully automated process for plasmid purification or a device or a system thereof.

EP 0 811 055 (Genzyme Corp) describes as its object to provide an automatable method for isolating high quality plasmids on a large scale to meet the future demand. More specifically, in a first aspect, EP 0 811 055 refers to a method for lysing cells. The method comprises simultaneously flowing a cell suspension and a lysis solution through a static mixer, wherein the cells exit the static mixer lysed. In another aspect, EP 0 811 055 refers to a method of simultaneously flowing a cell lysate or other protein-containing solution and precipitating solution through a static mixer, wherein the lysate or protein solution exits the static mixer with its precipitable components precipitated. The two methods may be combined by using static mixers in series. In an embodiment presented as preferred, suitable static mixers contain an internal helical structure which causes two liquids to come in contact with one another in an opposing rotational flow causing the liquids to mix together in a turbulent flow. One stated advantage of EP 0 811 055 is that large volumes of cells can be gently and continuously lysed in-line, greatly simplifying the process of isolating plasmids from large volumes of material without damaging plasmid DNA.

U.S. Pat. No. 5,330,914 (CEMU Bioteknik) relates to a method for automatic purification of extra-chromosomal DNA such as plasmids from a cell suspension, which comprises
introducing the cell suspension into a chamber,
introducing a lysing solution into the chamber and mixing it with the contents thereof to lyse the cells therein,
introducing a precipitating solution into the chamber and mixing it with the contents thereof to precipitate chromosomal DNA and, optionally, proteins and cell debris therein,
filtering the contents of the chamber through a filter and feeding the liquid contents to a collecting device,
purifying extra-chromosomal DNA from the contents of the collecting device,
introducing a dissolving solution into the chamber and mixing it with the contents thereof to dissolve the precipitate remaining therein, and
feeding the dissolved precipitate from the chamber to waste.

A mixing chamber is described as provided with agitating means, e.g. vibrating or rotating means, for mixing the cell suspension and the lysing solution.

US 2005/0026177 (Boehringer Ingelheim) relates to a scalable process and a device for producing a biomolecule, in particular pharmaceutical grade plasmid DNA. One object of US 2005/0026177 is to provide an automatable and scalable process for isolating a polynucleotide of interest, in particular plasmid DNA, on a manufacturing scale that includes, as a cell disintegration step, an improved alkaline lysis method. US 2005/0026177 describes how several mixing techniques were tested in preliminary experiments, after which it was found that a tube filled with glass beads leads to sufficient mixing and contacting of two solutions consisting of a cell suspension and a lysis solution, respectively. Further, US 2005/0026177 teaches that the described process may be run continuously and fully automated.

WO 2012/134440 (Gjerde et al) relates to methods and devices for sample preparation, such as separating, extracting or purifying nucleic acids, such as DNA and RNA, and more particularly plasmids. The method is described as highly automatable and intended for purifying nucleic acids in a pipette tip column format. However, it is clear from WO 2012/134440 that the term ‘automatable’ as used therein refers to the purification procedure that begins with the cell pellet, immediately after the lysis step.

Despite the existing methods, there is still a need for an automatable method for plasmid purification, which method provides for a small or at least reduced, compared to prior art methods, risk of shearing of genomic DNA and thereby contaminating the final product, e.g. an extracted or purified plasmid. In such automatable method, preferably all steps are automated or at least the important step of mixing cells with a lysis buffering agent is automated. There is also a related need for the actual hardware that can enable such method, for example a novel mixer which may gently treat delicate plasmids.

Similarly, there are many different ways to purify viral vectors or viral particles, but most of the existing processes suffer from drawbacks. One of such drawbacks is lack of the scalability that is required to purify large quantities of the vector or part thereof. Another drawback is that the prior art methods and process require numerous inefficient steps, thereby rendering them cost prohibitive and less productive due to cumulative yield losses. While many processes from well-known protein purification methods are adapted for viral particle purification, the complexity of viral particles provide for unique challenges that need to be addressed.

As far as purification of exosomes is concerned, one of the most pressing issues relate to the risk to damage vesicles during purification with loss of biological activity, the need of a sufficiently large sample and the efficiency of isolation.

### SUMMARY

In the light of the above, it is an object of the present invention to provide an automatable method for mixing two or more ingredients, an automatable process for purifying macromolecules and an automated system comprising a workstation configured for purifying macromolecules, as defined in the appended claims. Said invention alleviates at least in part the above-discussed problems and at least partially addresses one or more of the above-mentioned needs. Further details and advantages will follow from the detailed disclosure below as well as from the dependent claims.

According to a first aspect of the invention an automated method is provided for chemical treatment of cells in a mixing chamber, wherein the automatable method comprises
providing cells,
providing a chemical agent for treating cells,
forming a liquid mixture comprising the cells and the chemical agent, and
mixing the liquid mixture by providing a back-and-forth liquid flow in the mixing chamber.

According to another aspect of the invention an automated process is provided for purifying macromolecules, which process comprises
providing cells,
providing a lysis buffering agent,
providing a precipitation agent,
forming a liquid mixture comprising the cells and the lysis buffering agent, mixing the liquid mixture by providing a back-and-forth liquid flow in the mixing chamber,
providing a non-precipitated liquid mixture by adding the precipitation agent to at least a portion of the liquid mixture,
providing a filtrate by passing at least a portion of the non-precipitated liquid mixture through at least one filter.

According to yet another aspect of the invention an automated system comprising a workstation configured for purifying macromolecules produced by cells is provided, wherein the workstation comprises at least
a mixing chamber configured to perform back-and-forth liquid flow,
a filter configured to perform gravity filtration, and
a liquid chromatography column configured to perform for a back-and-forth liquid flow.

These and other aspects of the invention are apparent from and elucidated with reference to the embodiments described hereinafter.

### BRIEF DESCRIPTION OF THE DRAWINGS

For exemplifying purposes, the invention will be described in closer detail in the following with reference to embodiments thereof illustrated in the attached drawings.

FIG. 1 schematically illustrates an overview of an automated purification process according to the invention. The automated purification process includes the steps from suspending cells to recovering a pellet.

FIG. 2 schematically exemplifies an arrangement of a mixer of the invention, wherein the mixer is configured for performing in an automated back-and-forth-flow mode.

FIG. 3A schematically illustrates how the back-and-forth flow mixing may be accomplished in a flow-up in a vertically arranged container constituting a mixing chamber.

FIG. 3B schematically illustrates how the back-and-forth flow mixing may be accomplished in a flow down in a vertically arranged container constituting a mixing chamber.

FIG. 4 schematically exemplifies an arrangement of an automated system comprising a workstation configured for purifying macromolecules produced by cells, wherein the workstation is of a carousel-type.

FIG. 5 schematically illustrates steps of an automated process for purifying macromolecules, which process is performed on a workstation of a carousel-type.

### DETAILED DESCRIPTION OF EMBODIMENTS

In the following detailed description, the technical terms and expressions are defined, and embodiments of the invention are described.

Generally, all terms and expressions used in the application text are to be interpreted according to the meaning commonly applied to them in the pertinent prior art unless explicitly defined otherwise herein.

As used in this specification and the appended claims, the singular forms “a”, “an” and “the” include plural referents unless the context clearly dictates otherwise. Thus, e.g. “a macromolecule” includes one or more macromolecules, “a plasmid” includes one or more plasmids, “a cell” includes one or more cells and the like.

The term “macromolecule” is understood within the scope of the invention to refer to any very large molecule, which is considerably larger than the ordinary molecule, which usually has a diameter of less than 10 angstroms (10-6 mm). The macromolecule is composed of much larger numbers of atoms than the ordinary molecule. The corresponding molecular weight of such macromolecule is on the order of 35 000. The biologically important proteins and nucleic acids are among many substances that are made up of macromolecules.

Further, for the purpose of the present application, the term “macromolecules” in certain instances also covers plasmids, viruses and parts thereof.

The term “lysis buffer” is understood within the scope of the invention to refer to any additive that is capable of lysing the cells with which it is combined. In other words, the “lysis buffer” is a buffer comprising a “lysis buffering agent”.

Similarly, the term “precipitation buffer” is used in a broad context and is understood within the scope of the invention to refer to a buffer comprising a “precipitation agent”. Thus, there is no requirement of any pH-buffering effect of the “precipitation buffer” as used herein; “precipitation buffer” is an ingredient capable of providing precipitation.

Both terms—“lysis buffering agent” and “precipitation agent”—are used in a conventional sense for the pertaining technical field.

The term “chamber” is understood within the scope of the invention to refer to an enclosed space within an apparatus or the like. The terms “mixing chamber”, “chamber” and “mixing column” are used interchangeably throughout the application text.

Illustrative embodiments of the invention are described below.

The present inventors have developed a novel mixer, sometimes herein denoted as an Automated Gentle Mixer (AGM), for purification of at least one type of macromolecules, such as plasmids and viruses. As it appears from the disclosure below, the invention pertaining to treating and preparing cells for purification stands alone. A person skilled in the art, however, would appreciate that an automated method and an instrument for automation according to the present invention would enable further automation of any purification process for at least one type of macromolecules.

Thus, one advantage of the use of the AGM is that it can be fully integrated into an automatable process for purification. Such automatable process may be scalable to any desired scale. The technology of the present invention is based on a back-and-forth flow process.

Another advantage of the present invention is that the novel mixer provides for a gentle mixing of components which may at times be very delicate, such as plasmids, e.g. supercoiled plasmids and virus particles. The gentle mixing is obtained by avoiding the commonly used harsh conditions of turbulent flow and/or mechanical stirrers. Instead, according to the present invention combined components, or ingredients, are mixed by “folding” thereof together. “Folding” is a term used herein to describe the process of combining components, or ingredients, together in gentle way, i.e. without stirring or agitating, to avoid creating a turbulent flow.

The present invention is based on an automated mixing provided by a laminar flow. A flow of a liquid enters a tube, flows through the tube and expands into a chamber. As the flow expands, the liquid and suspended particulate flow back onto itself, i.e. the folding back causes mixing. A downward flow is also a laminar flow, wherein the folding back of the liquid and particulate takes place in the mixing chamber. An advantage of the present invention is that the mixing process therein is mechanical but gentle, scalable and integratable into a fully automated process for purification of macromolecules.

According to a first aspect of the invention an automated method is provided for chemical treatment of cells in a mixing chamber, wherein the automatable method comprises
providing cells,
providing a chemical agent for treating cells,
forming a liquid mixture comprising the cells and the chemical agent, and
mixing the liquid mixture by providing a back-and-forth liquid flow in the mixing chamber.

A person skilled in the art will appreciate that the above “liquid mixture” may also be considered a suspension, i.e. a heterogeneous mixture of a fluid that contains solid particles. By providing the back-and-forth liquid flow the liquid mixture, or the suspension, is being subjected to mixing, which mixing may provide for a more uniform liquid mixture, or a more uniform suspension.

According to the above automatable method two or more ingredients may be mixed in a mixing chamber. A first ingredient may be a liquid which comprises cells, and a second ingredient may be either a lysis buffer or a precipitation buffer. The mixing may be obtained by providing a back-and-forth liquid flow within the mixing chamber.

The method may further comprise introducing the liquid mixture into a tube, the tube being configured to be connectable to a mixing chamber, the mixing chamber being broader than the tube. A person skilled in the art would also realise that the above tube and the mixing chamber do not have to be separate entities. The liquid mixture may well be introduced into a passage, wherein the liquid mixture may first enter the first part of the passage and then the second part of the passage, the first part of the passage being broader than the second part of the passage.

The back-and-forth liquid flow may be provided at least by injecting liquid in the mixing chamber by generating a pressure gradient using a pump configured to be connectable to the mixing chamber. Applying vacuum to the mixing chamber may lead to aspiration of liquid into the mixing chamber. The movement of the liquid in the mixing chamber may be described as a back-and-forth liquid flow. A person skilled in the art would realise that “vacuum” in this context is not to be understood in absolute terms, but instead to be understood in relative terms.

Alternatively, the back-and-forth liquid flow may be provided at least by ejecting liquid from the mixing chamber by generating a pressure gradient using a pump configured to be connectable to the mixing chamber. Applying a pump action to the mixing chamber may lead to dispensing of the liquid contained therein. The movement of the liquid may be described as a back-and-forth liquid flow.

The direction of the pressure gradient may alternate a number of times, preferably 2-10 times, more preferably 4-6 times. The back-and-forth flow mode may be obtained by repeating aspiration in and dispensing from the mixing chamber any number of times, such as 1-10 times, e.g. 2-5 times.

The back-and-forth flow mode is a well-known principle primarily in chromatography, where it is sometimes known as dual flow chromatography, see e.g. U.S. Pat. No. 7,482,169 (Gjerde et al). Therefore, a person skilled in the art can easily configure commercially available hardware to perform the claimed mixing using the back-and-forth flow mode.

The present invention is based on the finding that the back-and-forth flow may be configured in a way that produces a fully satisfactory mixing of liquid components across protocols for the purification of macromolecules, such as plasmids or virus. One major advantage of employing this flow is that it renders such protocols fully automatable by providing an opportunity for integrating the automatic mixing according to the present invention into any automated prior art process. The above allows to avoid the manual and consequently time-consuming careful turning of containers. Up to now such turning of the containers has been required to achieve a fully satisfactory mixing of liquid components across protocols for the purification of macromolecules.

Most conveniently, each ingredient is introduced into a tube, after which they are added to the top or upper part of the mixing chamber, allowing them to expand therein. The mixing chamber, sometimes herein denoted as the expansion chamber, may be arranged in any suitable container. One advantage of the invention is that the mixer is easily disposed of. In addition, cleaning thereof is easier than that of prior art equipment, because the mixer of the invention may stand alone and not be integrated into valves tubing etc.

As the skilled person will appreciate, the volume of the expansion chamber is advantageously large enough to process the entire sample that is being mixed.

For large scale applications, it may be appropriate to use a column volume of 1 L (Giga scale) with a bed volume of 25 mL or a column volume of 10 L (Tera scale) with a bed volume of 259 mL.

In an illustrative example, the mixer volume may be about 1 L, while the tube dimensions are about 4-8 mm i.d. tube. The person skilled in the art will appreciate that the size of the tube may be adjusted to the size of the mixing chamber. Smaller tubes may be used for smaller mixing chamber for smaller plasmid preparations. Larger tubes may be used for larger mixing chambers for larger plasmid preparations. To this end, standard tubing and other equipment may be easily selected and adapted as appropriate by the skilled person. For example, the dimensions of tubing and/or mixing chambers may be selected to provide the desired degree of laminar liquid flow, for example, a liquid flow which is almost entirely laminar.

Thus, in an advantageous example, the conditions, hardware and settings are chosen or configured to provide for a substantially laminar flow within the mixing chamber and optionally within the other parts of the set-up used. The present inventors unexpectedly found that the gentler conditions of laminar flow mixing are efficient for the mixing of liquids, such as cell suspensions, the contents of which may be damaged when using prior art protocols utilising a harsher turbulent flow, e.g. as the one obtained with mechanical stirring means.

In one example, the flow within the mixing chamber will allow for some turbulent flow.

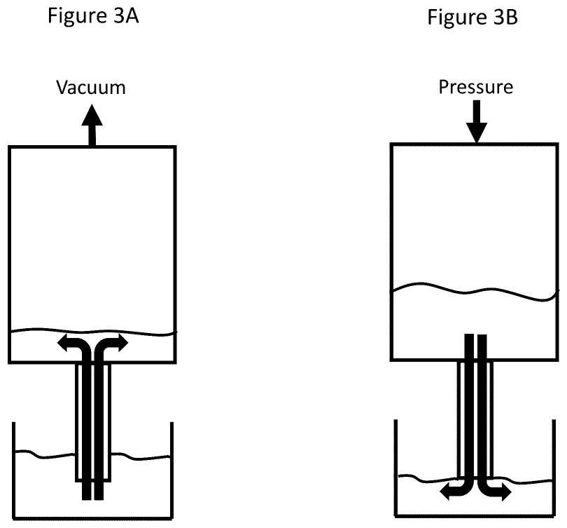

In an advantageous example, as illustrated in FIG. 3A and FIG. 3B, respectively, vacuum is arranged to provide for aspiration of liquid upwards, while pumping may provide for dispensing of liquid downwards. Gentle mixing occurs in both directions. Any suitable equipment, such as a peristaltic pump, may be used to this end. As the skilled person will appreciate, the way the back-and-forth flow is accomplished may be obtained in many different ways, depending e.g. on the desired degree of mixing as well as the nature of the liquid comprising cells, sometimes herein denoted the “cell liquid”, and therefore it is not a limiting feature of this invention.

The cells may be provided in form of a cell suspension. The cells may be provided in form of a cell colony on the surface of or within a solid medium, such as a growth medium.

The liquid which comprises cells, i.e. the cell liquid, such as a cell suspension, may have been subjected to one or more steps of pre-treatment and/or conditioning before being added to the mixing chamber of the invention. Such preceding steps may e.g. be a pH-conditioning, centrifugation, decantation of some liquid or any other appropriate measure which is commonly used in the field.

The chemical agent may be a lysis buffering agent or a lysis buffer. Advantageously, the liquid mixture may comprise a DNase or a RNase, or the lysis buffer may comprise a DNase or a RNase.

According to the automated method of the present invention at least a portion of the cells comprised within the liquid mixture may undergo lysis in the mixing chamber During the lysis the cells release macromolecules, such as virus and/or plasmids. At least a portion of the cells of the liquid mixture release macromolecules.

If a lysis buffer is mixed with the cell liquid, the cells advantageously undergo lysis within the mixing chamber. The duration of such lysis step may be adjusted due to factors well known to the skilled person in this field, such as the cell type and the lysis agent, the liquid flow rate etc. Such cells may be eukaryotic or prokaryotic cells, such as bacterial cells. Advantageous cells are recombinant cells which have been produced to express certain genes or proteins which in their purified form are useful in pharmaceutical applications such as therapy or vaccines. Thus, the lysed cells may release macromolecules, such as virus and/or plasmids. Advantageously, the released macromolecules are plasmids, such as a supercoiled plasmid DNA.

The chemical agent may be a precipitation agent or a precipitation buffer.

The mixing of the invention can be used for any type of lysis and precipitation sample preparation chemistry, including preparation for chaotropic plasmid purification or ion exchange plasmid purification. The ion exchange plasmid purification, including anion exchange plasmid purification, may be as described in any of the following international publications WO 2022/076543 and WO 2021/170564, which are incorporated by reference herein.

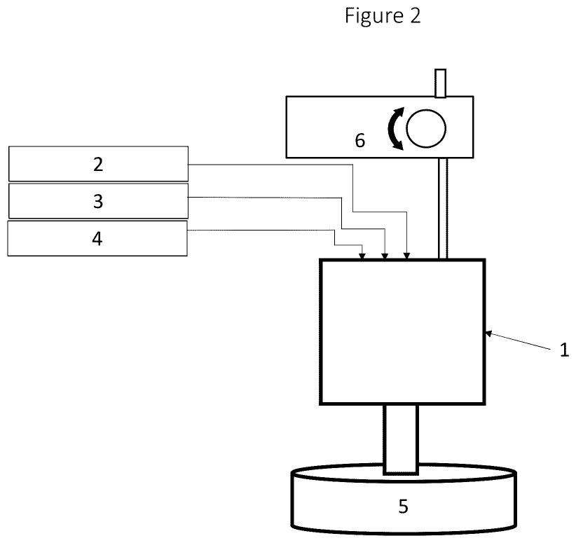

In addition to the cell liquid and lysis buffer and/or precipitation buffer, the present method may also comprise at least one additional ingredient, such as a wash buffer(s). Such additional ingredients may be reagents which are pumped to the mixing chamber separately, as illustrated in FIG. 2. The skilled person can conclude using the common general knowledge what additional ingredients are suitable depending e.g. on the cells as well as on the desired macromolecule to be purified.

Further, various ingredients may be added to the mixing chamber after the initiated mixing, i.e. after one or more cycles of the back-and-forth flow. For example, after a first period or cycle of mixing e.g. the mixing of a liquid mixture comprising cells and a lysis buffering agent, a precipitation buffer or a precipitation agent may be added into the mixing chamber.

According to another aspect of the invention an automated process is provided for purifying macromolecules, which process comprises
providing cells,
providing a lysis buffering agent,
providing a precipitation agent,
forming a liquid mixture comprising the cells and the lysis buffering agent, mixing the liquid mixture by providing a back-and-forth liquid flow in the mixing chamber,
providing a non-precipitated liquid mixture by adding the precipitation agent to at least a portion of the liquid mixture,
providing a filtrate by passing at least a portion of the non-precipitated liquid mixture through at least one filter.

The present invention also relates to an automatable process for purifying macromolecules from a liquid comprising cells, which process comprises mixing the cell liquid at least with either a lysis buffer or a precipitation buffer in back-and-forth liquid flow mode within a mixing chamber to produce a liquid mixture including lysed and/or precipitated cells. Once mixed, the lysed or precipitated cells may be subjected to any commonly used further step for purification, such as with help of a membrane, bead, fibre or column step. Optionally, said liquid mixture including lysed and/or precipitated cells may be passed through at least one filter to produce a filtrate.

The filter may be any suitable filter which is automatable and capable of separating macromolecules from other components present in the liquid that exits the mixing chamber, such as a gravity filter. Examples of components separated by the filtering step are cell debris, flocculants and certain larger proteins. Though the process of the invention may include more than one filter without deviating from the scope and spirit of the invention, an advantageous and automatable example comprises a single step of filtration, which is based on the principle of gravity filtration. In order to maximise the yield of macromolecules, the filtering step may include washing of the filter by addition of a suitable wash liquid after passage of the mixed liquid through the filter.

The above automated process may further comprise capturing macromolecules by passing at least a portion of the filtrate through a chromatography column and eluting at least a portion of the macromolecules from the chromatography column.

The chromatography column may be any suitable column capable of separating macromolecules from other components present in the filtered liquid, such as any still remaining cell debris or flocculants as well as smaller proteins. For example, a chromatography column can be configured to selectively separate desired macromolecules from endotoxins, undesired virus or parts thereof. Equipment for this step is commercially available on the market and may easily be adapted by the skilled person for use according to the present invention.

In an advantageous example, the passing of the liquid across a chromatography column includes back-and-forth flow. As discussed above, such flow is well known in the field of chromatography, and the skilled person can easily choose optimal conditions for a devised separation.

The above automated process may further comprise concentrating the macromolecules, including the macromolecules eluted from the chromatography column; i.e. the process may comprise a step of concentration to produce a solid precipitate which includes purified macromolecules. So-called ethanol precipitation or (ethanol) column concentration may be a last step of the process. At this step, together with ethanol, a salt at a high concentration, usually potassium or sodium acetate, may be added.

As discussed above, one major advantage of the present invention is that it enables automated mixing of cell liquids with other ingredients such as a lysis buffer. Thus, in one example of the present process, at least the mixing, and also preferably the filtering and the step(s) employing chromatography, are configured to be automated.

According to yet another aspect of the invention an automated system comprising a workstation configured for purifying macromolecules produced by cells is provided, wherein the workstation comprises at least
a mixing chamber configured to perform back-and-forth liquid flow,
a filter configured to perform gravity filtration, and
a liquid chromatography column configured to perform for a back-and-forth liquid flow.

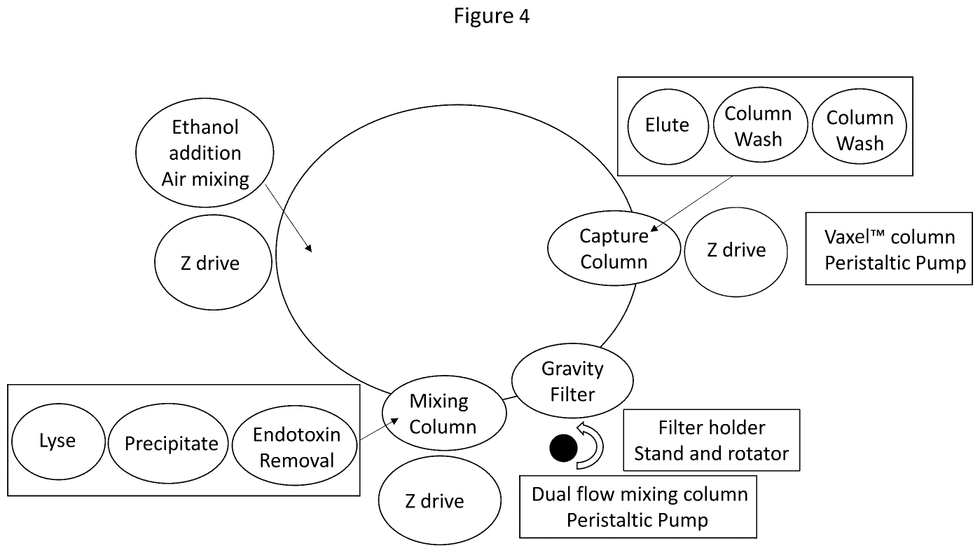

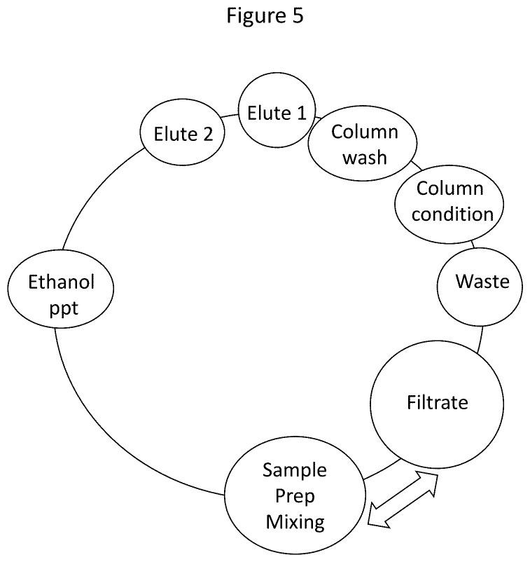

In an advantageous example of the system of the invention, the workstation is a carousel, i.e. a system enabling for continuous circular processing of a cell culture liquid to purify desired macromolecules, such as plasmids or virus. One example of such system including a carousel-type workstation is illustrated in FIGS. 4 and 5.

The system according to the invention may also comprise containers holding liquids, such as an eluent liquid and a wash liquid. The system may be configured for a purification at a scale of GigaPrep or TeraPrep, which terms are commonly used and well known in this field.

The modules of the system may be Z drive mounted, in accordance with well-known principles. Advantageously, the workstation is designed for a specific use to fully benefit from all advantages while optimizing the speed of processing. For example, a 2-tier carousel may be used, which is top level stable and bottom level mobile.

It is appreciated that the above-mentioned embodiments of the first aspect of the present invention may also apply, even if not explicitly mentioned, to the further aspects of the present invention.

The invention has mainly been described above with reference to some embodiments. However, as it is readily appreciated by the person skilled in the art, other embodiments than the ones disclosed above are equally possible within the scope of the invention as defined by the appended claims.

### DETAILED DESCRIPTION OF THE DRAWINGS

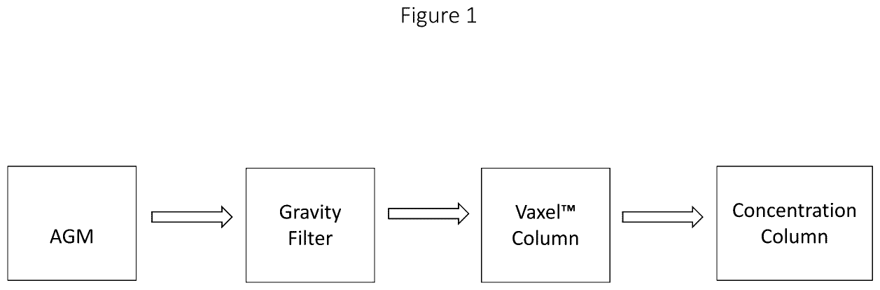

FIG. 1 schematically shows an overview of an automated purification process according to the invention. The automated purification process covers the steps from suspending cells to recovering a pellet. This overview exemplifies how a mixing step(s) may be integrated into a fully automated plasmid purification process. More specifically, the process starts with the application of an automated gentle mixer (AGM) mounted on Z drive. Afterwards a gravity filter is applied. Then, a Z drive mounted chromatography column, such as a Växel™ column (Biotage AB), is applied. The Z drive mounted chromatography column configured to enable capturing macromolecules as well as washing thereof before elution. Finally, a Z drive mounted column for concentrating with help of ethanol may optionally be used.

FIG. 2 schematically exemplifies an arrangement of a mixer of the invention, wherein the mixer is configured for performing in an automated back-and-forth-flow mode. Individual ingredients, such as Reagent A, Reagent B and Reagent C, are all provided separately to the mixing chamber 1 by respective reagent pumps, i.e. by Reagent pump A 2, Reagent pump B 3 and Reagent pump C 4. Suspended cells 5 are also provided. Gentle mixing occurs in both directions. To this end, a peristaltic pump 6 is used.

FIG. 3A schematically illustrates how the back-and-forth flow mixing may be accomplished in a flow-up in a vertically arranged container constituting a mixing chamber.

FIG. 3B schematically illustrates how the back-and-forth flow mixing may be accomplished in a flow down in a vertically arranged container constituting a mixing chamber.

FIG. 4 schematically exemplifies an arrangement of an automated system comprising a workstation configured for purifying macromolecules produced by cells, wherein the workstation is of a carousel-type. The workstation includes GigaPREP and TeraPREP Stationary Towers and containers as buffer reservoirs. Växel™ chromatography columns are used in the exemplary workstation. Consumables for the processes of lysis, precipitation, endotoxin removal, aqueous filtrate wash and elution are bulk.

FIG. 5 schematically illustrates steps of an automated process for purifying macromolecules, which process is performed on a workstation of a carousel-type, more specifically on GigaPREP and TeraPREP Carousel Rotating Stations. The process steps include mixing and filtration followed by column chromatography, column wash before two subsequent steps of selective elution and concentration with use of ethanol.

### Examples

The present examples are provided for illustrative purposes only and should not be construed to be limiting the invention as defined by the appended claims. All references provided below and elsewhere in the present application are hereby included herein via reference.

### Example 1: Plasmid Purification-Fully Automated Sample Flow

To carry out the below process, a commercially available workstation adaptable to the required hardware elements may be used. The protocol may be read as follows:
1. Resuspend 15 g cell pellet. Add glass beads, RNase and dye. 87 mL shake. Pour into a sample container in the instrument. It is possible to automate the cell pellet resuspension with help of a dual flow mixing column, this step, however, requires rigorous mixing. The dual flow mixing column is designed for gentle mixing, and this technology may be better suited for use in gentle lysing and precipitating to prevent genomic DNA shearing.
2. Pump in lysis buffer, 103 mL and dual flow mix.
3. The column head pressure, vacuum and pumping volumes are controlled by a peristaltic pump attached at the top of the column.
4. Pump in precipitation buffer, 123 mL dual flow mix.
5. Pump in 25 mL ERB endotoxin removal buffer, very gentle dual flow mix, one cycle.
   Notes: Try to avoid bubble formation and shearing of genomic DNA. ERB is a surfactant. Pump to the top of a dual flow mixing column. For each bulk reagent, pumps are peristaltic. Consumables for the processes of lysis, precipitation, ERB removal and possible aqueous filtrate wash are bulk.
6. Aspirate the mixture into the dual flow mixing column and rotate carousel (FIGS. 4 and 5) clockwise to position a gravity filter with a processing reservoir under the dual flow mixing column. Recover 300 mL filtrate.
   Notes: operations 5 and 6 are performed at the same time.
7. Equilibrate a 20 mL bed column with 60 mL equilibration buffer. Pump 60 mL into the top of the column and dual flow mix. Dispense.
   Notes: The filtrate is still under filter, and the column is at the conditioning position with an equilibration reservoir below.
8. Rotate counterclockwise to position the filtrate sample under a capture column. Capture entire sample with 8 dual flow cycles.
   Notes: The column head pressure and vacuum are controlled by a peristaltic pump positioned at the top of the column.
9. Rotate the carousel clockwise to a wash sample reservoir, pump in 200 mL and dual flow wash.
   Notes: Washing may be repeated. Washing may be a top-down flow.
10. Rotate the carousel clockwise to an elute reservoir. Pump in 60 mL and dual flow elute. Use a 200 mL plastic capture bottle.
   Notes: Eluting may be a top-down flow.
11. Capture of the same sample may be repeated. For every capture cycle, an additional wash position and an elution position may be needed. In this example, 3 elution positions have been utilised.
12. Rotate the carousel clockwise and pump in ethanol to carry out a precipitation step. Insert tube to pump ethanol. Use bubble mixing.

1. An automated method for chemical treatment of cells in a mixing chamber, wherein the automatable method comprises
providing a mixing chamber,
providing cells,
providing a chemical agent for treating cells,
combining said cells and said chemical agent in the mixing chamber to form a liquid mixture, and
providing for mixing of said cells and said chemical agent by providing a back-and-forth liquid flow in the mixing chamber.

2. The method according to claim 1, wherein the method further comprises introducing the liquid mixture into a tube, the tube being configured to be connected to the mixing chamber, the mixing chamber being broader than the tube.

3. The method according to claim 1, wherein the back-and-forth liquid flow is provided at least by injecting liquid in the mixing chamber by generating a pressure gradient using a pump configured to be connected to the mixing chamber.

4. The method according to claim 1, wherein the back-and-forth liquid flow is provided at least by ejecting liquid from the mixing chamber by generating a pressure gradient using a pump configured to be connected to the mixing chamber.

5. The method according to claim 3, wherein the direction of the pressure gradient alternates a number of times, preferably 2-10 times, more preferably 4-6 times.

6. The method according to claim 1, wherein the cells are provided in form of a cell suspension.

7. The method according to claim 1, wherein the chemical agent is a lysis agent.

8. The method according to claim 7, wherein the liquid mixture comprises a DNase or a RNase.

9. The method according to claim 7, wherein at least a portion of the cells comprised within the liquid mixture undergoes lysis in the mixing chamber.

10. The method according to claim 7, wherein at least a portion of the cells of the liquid mixture release macromolecules.

11. The method according to claim 1, wherein the chemical agent is a precipitation agent.

12. An automated process for purifying macromolecules, which process comprises
providing cells,
providing a lysis buffering agent,
providing a precipitation agent,
forming a liquid mixture comprising the cells and the lysis buffering agent, mixing the liquid mixture by providing a back-and-forth liquid flow in the mixing chamber,
providing a non-precipitated liquid mixture by adding the precipitation agent to at least a portion of the liquid mixture,
providing a filtrate by passing at least a portion of the non-precipitated liquid mixture through at least one filter.

13. The process according to claim 12, wherein the process further comprises
capturing macromolecules by passing at least a portion of the filtrate through a chromatography column,
eluting at least a portion of the macromolecules from the chromatography column.

14. The process according to claim 13, wherein the process further comprises concentrating the macromolecules eluted from the chromatography column.

15. The process according to claim 12, wherein the filter is a gravity filter.

16. The process according to claim 13, wherein the passing of the liquid across a chromatography column includes back-and-forth liquid flow.

17.-19. (canceled)
